(19) United States
(12) Patent Application Publication
(10) Pub. No.: US 2010/0016269 A1
(43) Pub. Date: Jan. 21, 2010

(54) COMPOSITION OF PLANT STEROL AND PHOSPHATIDYLCHOLINE AND METHOD FOR PRODUCING THE SAME

(75) Inventors: Mitsugu Furukawa, Kanagawa (JP); Takao Makino, Kanagawa (JP); Masao Nakadate, Yamanashi (JP)

Correspondence Address: LUCAS & MERCANTI, LLP, 475 PARK AVENUE SOUTH, 15TH FLOOR, NEW YORK, NY, 10016, US

(73) Assignee: TAMA BIOCHEMICAL CO., LTD., Tokyo (JP)

(21) Appl. No.: 12/519,642

(22) PCT Filed: Jun. 5, 2007
(86) PCT No.: PCT/JP2007/061681

§ 371 Date: Jun. 17, 2009

(30) Foreign Application Priority Data
Dec. 26, 2006 (JP) 2006-350074

(51) Int. Cl. A61K 31/575; A61P 3/00

(52) U.S. Cl. 514/182

(57) ABSTRACT

The present invention is intended to discover a composition that contains a large amount of a plant sterol completely insoluble in water and exhibiting poor solubility in oil because of its extremely high crystallinity. Such composition can be easily dispersed in water, and to produce the composition. It has been discovered that a composition obtained by adding and dissolving a small amount of phosphatidylcholine in a plant sterol that is hardly soluble in water or in oil results in such composition easily dispersible in water.

### TECHNICAL FIELD

The present invention relates to a composition of a plant sterol and phosphatidylcholine, which contains 80% by weight or more and is easily dispersed in water and to a method for producing the composition.

### BACKGROUND ART

The people of Japan today are said to tend to excessively ingest cholesterol because of a westernized dietary habit, diversification of lifestyle owing to changes in the social environment, or the like. Hence, Japanese are increasingly affected by diseases such as hyperlipidemia, arteriosclerosis, myocardial infarction, and arrhythmia, which are caused by excessive blood cholesterol levels.

Plant sterols are known to suppress the in vivo absorption of cholesterol that is excessively ingested as described above, so that they are currently attention-attracting materials.

Studies concerning the cholesterol-lowering effect of a plant sterol began from an experiment using animals conducted in 1953 at the University of California, Berkeley (Proc. Soc. Exptl. Biol. Med. 83, 498-9 (1953) (Non-Patent Document 1) and J. Nutrition 50, 191-201 (1953) (Non-Patent Document 2)). Moreover, such effect has also been confirmed in an experiment using humans (Circulation 7, 702-6 (1953) (Non-Patent Document 3)).

In 1974, “Anti-Cholesterol Agent” (U.S. Pat. No. 3,852,440 (Patent Document 1)) was patented and commercialized in the U.S.A., so that the effectiveness became widely known.

In recent years, studies concerning the effects of a plant sterol on cholesterol have been reported, such as a greater number of detailed clinical trials (Br. J. Nutr. 86 (2), 233-9 (2001) (Non-Patent Document 4)) and a mechanism for the inhibition of cholesterol absorption (Atherosclerosis 160 (2), 477-81 (2002) (Non-Patent Document 5)). Thus, the effectiveness has been confirmed.

However, plant sterols are completely insoluble in water and poorly soluble in oil because of their extremely high crystallinity. Accordingly, the use of such plant sterols for foods, beverages, and the like required ingenuity.

Examples of patents that have been reported, by which plant sterols are formulated so that they can be easily dispersed in water for use in foods, beverages, or the like, include the patent using a monoglyceride that is an emulsifier (JP Patent Publication (Kokai) No. 6-329588 A (1994) (Patent Document 2)), the patent using a monoglyceride and polysorbate that are also emulsifiers (JP Patent Publication (Kohyo) No. 2005-521397 A (Patent Document 3)), and the patent using polysorbate and dextrin (JP Patent Publication (Kokai) No. 2005-269941 A (Patent Document 4)), for example. However, with the use of monoglyceride alone, only a maximum of 40% by weight of a plant sterol is contained in a preparation. Moreover, regarding the use of polysorbate, it is an emulsifier and the use of such polysorbate is prohibited in foods in Japan.

Also, examples of patents that have been reported include the patent using emulsifiers such as a glycerine fatty acid ester, a sorbitan fatty acid ester, a propylene glycol fatty acid ester, a sucrose fatty acid ester, and a polyglycerine fatty acid ester, and fats and oils that are liquids at room temperature such as lipids (e.g., medium chain triglyceride, squalene, and squalane (JP Patent No. 1938786 (Patent Document 5)) and the patent using emulsifiers such as a glycerine fatty acid ester, organic acid monoglyceride, a polyglycerine fatty acid ester, a propylene glycol fatty acid ester, and a sucrose fatty acid ester, edible oils, at least one type of hydrophilic polyglycerine fatty acid ester, and water (JP Patent Publication (Kokai) No. 2002-291442 A (Patent Document 6)). They are also problematic in that only a 50% by weight to 60% by weight at maximum, of a plant sterol can be contained in a preparation.

Furthermore, according to the patent using lecithin, fats and oils, polyhydric alcohol, and ethanol (JP Patent Publication (Kokai) No. 2001-117 A (Patent Document 7)), a plant sterol can be dispersed in water, but the content of the plant sterol is up to 15% by weight. The patent using oil, free fatty acid, and phospholipid (JP Patent Publication (Kohyo) No. 2004-534793 A (Patent Document 8)) and the patent using fat, water, salt, and lecithin (JP Patent Publication (Kohyo) No. 2005-524396 A (Patent Document 9)) are patents by which plant sterols are used for foods and the like via technology for easily dispersing the plant sterols in fats and oils. Such technology concerning the application of plant sterols to foods and the like with the use of lecithin is employed for increasing the solubility of plant sterols in fats and oils or for suppressing the crystallinity of plant sterols in fats and oils in many patents.

Patent Document 1 U.S. Pat. No. 3,852,440

Patent Document 2 JP Patent Publication (Kokai) No. 6-329588 A (1994)

Patent Document 3 JP Patent Publication (Kohyo) No. 2005-521397 A

Patent Document 4 JP Patent Publication (Kokai) No. 2005-269941 A

Patent Document 5 JP Patent No. 1938786

Patent Document 6 JP Patent Publication (Kokai) No. 2002-291442 A

Patent Document 7 JP Patent Publication (Kohyo) No. 2001-117 A

Patent Document 8 JP Patent Publication (Kohyo) No. 2004-534793 A

Patent Document 9 JP Patent Publication (Kohyo) No. 2005-524396 A

Non-Patent Document 1 Proc. Soc. Exptl. Biol. Med. 83, 498-9 (1953)

Non-Patent Document 2 J. Nutrition 50, 191-201 (1953)

Non-Patent Document 3 Circulation 7, 702-6 (1953)

Non-Patent Document 4 Br. J. Nutr. 86 (2), 233-9 (2001)

Non-Patent Document 5 Atherosclerosis 160 (2), 477-81 (2002)

### DISCLOSURE OF THE INVENTION

An object of the present invention is to discover a composition that contains a large amount of a plant sterol that is completely insoluble in water and is poorly soluble in oil because of its extremely high crystallinity and can be easily dispersed in water and to produce the composition.

As a result of intensive studies to achieve the above object, the present inventors have discovered that a composition obtained by adding and dissolving a small amount of phosphatidylcholine in a plant sterol hardly soluble in water and oil can be easily dispersed in water, and thus they have completed the present invention.

The present invention relates to (1) to (5).

(1) A composition easily dispersible in water, which is obtained by dissolving phosphatidylcholine in a plant sterol.
(2) The composition according to (1), wherein a sucrose fatty acid ester is added.
(3) The composition according to (1) or (2), wherein the content of the plant sterol is 80% by weight or more.
(4) The composition according to (1) or (2), wherein the content of the plant sterol is 95% by weight or more.
(5) A method for producing a composition that is easily dispersed in water and comprises a plant sterol and phosphatidylcholine, which comprises the following [1] or [2] of:
[1] heating and melting a plant sterol in advance and subsequently adding and dissolving phosphatidylcholine; or
[2] dissolving a plant sterol and phosphatidylcholine in an organic solvent in which both plant sterol and phosphatidylcholine can be dissolved, subsequently distilling off the organic solvent, and then conducting drying.
(6) A method for producing a composition that is easily dispersed in water and comprises a plant sterol, phosphatidylcholine, and a sucrose fatty acid ester, which comprises the following [1] or [2] of:
[1] heating and melting a plant sterol in advance and subsequently adding and dissolving phosphatidylcholine and a sucrose fatty acid ester; or
[2] dissolving a plant sterol, phosphatidylcholine, and a sucrose fatty acid ester in an organic solvent in which they can be dissolved together, subsequently distilling off the organic solvent, and then conducting drying.

The present inventors have found from conventional documents, patents, and the like that lecithin is a substance effective for dispersing a plant sterol in water and examined it. However, the present inventors have discovered that in the case of a preparation in which the percentage of a plant sterol to the preparation is increased, results in poorer dispersibility in water leading to the generation of a large amount of insoluble matter.

The reason is considered that lecithin is a mixture of phospholipids, fats and oils, and the like. Hence, the present inventors have conducted examination by varying the types of phospholipids such as purified phosphatidylcholine, phosphatidylglycerol, phosphatidylserine, phosphatide acid, and phosphatidyl ethanolamine. The present inventors have discovered that specifically a composition of phosphatidylcholine and a plant sterol is easily dispersed in water, despite of the high ratio of the plant sterol in the composition. Thus, the present inventors have completed the present invention.

The method for producing a composition easily dispersible in water of the present invention is preferably a method that involves heating and melting a plant sterol in advance and subsequently adding and dissolving phosphatidylcholine or a method that involves dissolving a plant sterol and phosphatidylcholine in an organic solvent in which they can be dissolved together, distilling off the organic solvent, and then conducting drying. As organic solvents, alcohols such as ethanol are preferred. However, types of such organic solvent are not limited, as long as they are organic solvents in which plant sterols and phosphatidylcholine can be dissolved. Specifically, a plant sterol is weighed in a container and then heated and melted. Phosphatidylcholine is added and dissolved and then the resultant is cooled to room temperature, so that it is solidified. The resultant is milled with a mill or the like, followed by sieving with a vibration sieving machine.

Furthermore, a method preferred when a sucrose fatty acid ester is added involves heating and melting a plant sterol in advance and subsequently adding and dissolving phosphatidylcholine and a sucrose fatty acid ester or involves dissolving a plant sterol, phosphatidylcholine, and a sucrose fatty acid ester in an organic solvent in which they can be dissolved together, subsequently distilling off the organic solvent, and then conducting drying.

To reveal water dispersibility when the present invention is implemented, experimental results (the state observed after 19 hours) were photographed and shown in FIG. 1. As can be seen from the images, No. 11 (plant sterol (98% by weight) and phosphatidylcholine (2% by weight)) was completely dispersed, but No. 47 (plant sterol (95% by weight) and a sucrose fatty acid ester (5% by weight)) partially remained undissolved on the bottom of the sample vial. Also, No. 76 (plant sterol (96.5% by weight), phosphatidylcholine (0.5% by weight), and a sucrose fatty acid ester (3% by weight)) was dispersed to almost the same extent as that in the case of No. 11.

In addition, in the present invention, “easily dispersed (or dispersible) in water” refers to a state in which the subject is easily dispersed in water, but is not dissolved.

This description includes part or all of the contents as disclosed in the description and/or drawings of Japanese Patent Application No. 2006-350074, which is a priority document of the present application.

### BRIEF DESCRIPTION OF THE DRAWINGS

FIG. 1 shows the results of separately dissolving No. 11 (Table 1, Example 3) and No. 76 (Table 1, Example 8) powders obtained according to the present invention and, as a control for comparison, No. 47 powders comprising a plant sterol (95% by weight) and a sucrose fatty acid ester (5% by weight) in high fructose corn syrup at a solid content concentration of 10% by weight and then allowing them to stand for 19 hours.

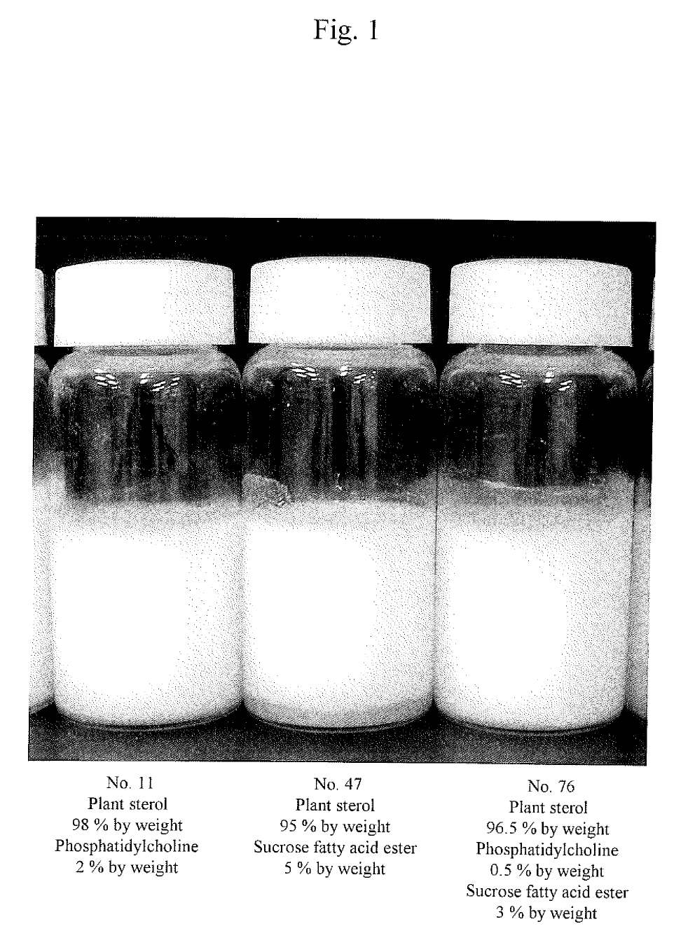

### BEST MODES FOR CARRYING OUT THE INVENTION

Hereafter, the present invention is described in greater detail.

A plant sterol to be used in the present invention is a sterol extracted and purified from, seeds such as soybean and rapeseed. Specifically such a plant sterol is one of or a mixture of two or more of brassicasterol, campesterol, stigmasterol, and β-sitosterol. The plant sterol is completely insoluble in water and has low solubility in oil.

Phosphatidylcholine to be used in the present invention is a type of phospholipid, which is a substance contained in lecithin obtained in a degumming step when fats and oils or the like are purified from soybean, yolk, rapeseed, flaxseed, or the like. In lecithin, in addition to phosphatidylcholine, phospholipids such as phosphatidylglycerol, phosphatidylserine, phosphatide acid, and phosphatidyl ethanolamine are mixed. Lecithin is further subjected to an extraction and purification step for a solvent such as ethanol, a step of column chromatography for purification, and the like. Lecithin, which is thus purified so that the content of phosphatidylcholine is 70% or more, is used.

A sucrose fatty acid ester to be used in the present invention is formed of sucrose and C14-18 saturated and unsaturated fatty acid, which is an emulsifier generally used for foods and the like. Examples of such esters include those going under trade names: Ryoto Sugar Esters S-1170, S-1570, and S-1670 produced by Mitsubishi-Kagaku Foods Corporation.

The reason for the addition of a sucrose fatty acid ester to the composition of a plant, sterol and phosphatidylcholine is that, via the addition thereof, the composition of the plant sterol and phosphatidylcholine, which is easily dispersed in water, can be prepared, even if the amount of phosphatidylcholine to be added is decreased.

The ratio of a plant sterol to phosphatidylcholine in terms of weight is preferably plant sterol:phosphatidylcholine=99:1 to 80:20 and more preferably plant sterol:phosphatidylcholine=99:1 to 95:5. When phosphatidylcholine is contained at a ratio lower than 99:1, the obtained composition of the plant sterol and phosphatidylcholine has poor dispersibility in water, so that insoluble matter floats on the liquid surface. Furthermore, when phosphatidylcholine is contained at a ratio higher than 80:20, the thus obtained composition of the plant sterol and phosphatidylcholine enters a tenacious state, so that it is problematic in terms of availability when added, mixed, and the like to foods and the like.

The thus obtained composition of a plant sterol and phosphatidylcholine can be dispersed well in water, although it contains the plant sterol as high as 80% by weight or more.

Such composition containing a plant sterol can be used according to its expected physiological functions since the plant sterol has a cholesterol-lowering effect.

Hereafter, the present invention is described in more detail with reference to Examples and Comparative Examples, although the present invention is not limited thereto.

### EXAMPLE 1

In accordance with the compositions listed in Table 1, 9.9 g of a plant sterol (Trade name: phytosterol FK, Tama Biochemical Co., Ltd.) was heated and melted and then 0.1 g of phosphatidylcholine (phosphatidylcholine content: 95.0%) was added and dissolved. The resultant was cooled and solidified and then milled with a small mill (HSIANG TAI MACHINERY INDUSTRY, Sample Mill SM-1), so that 10 g of white powder was obtained.

The thus obtained powder comprised brassicasterol (5.6%), campesterol (24.5%), stigmasterol (20.0%), and β-sitosterol (45.2%). The powder was dispersed in water to a solid content concentration of 1 w/v % and then the resulting state was observed. The results are shown in Table 1.

### EXAMPLE 2

In accordance with the compositions listed in Table 1, procedures were carried out in a manner similar to that in Example 1 except that the amount of the plant sterol added was 9.85 g and the amount of phosphatidylcholine added was 0.15 g. Thus, 10 g of white powder was obtained.

The thus obtained powder comprised brassicasterol (5.6%), campesterol (24.3%), stigmasterol (19.9%), and β-sitosterol (45.0%). The powder was dispersed in water to a solid content concentration of 1 w/v % and then the resulting state was observed. The results are shown in Table 1.

### EXAMPLE 3

In accordance with the compositions listed in Table 1, procedures were carried out in a manner similar to that in Example 1 except that the amount of the plant sterol added was 9.8 g and the amount of phosphatidylcholine added was 0.2 g. Thus, 10 g of white powder was obtained.

The thus obtained powder comprised brassicasterol (5.6%), campesterol (24.2%), stigmasterol (19.8%), and β-sitosterol (44.8%). The powder was dispersed in water to a solid content concentration of 1 w/v % and then the resulting state was observed. The results are shown in Table 1.

### EXAMPLE 4

In accordance with the compositions listed in Table 1, procedures were carried out in a manner similar to that in Example 1 except that the amount of the plant sterol added was 9.5 g and the amount of phosphatidylcholine added was 0.5 g. Thus, 10 g of white powder was obtained.

The thus obtained powder comprised brassicasterol (5.4%), campesterol (23.5%), stigmasterol (19.2%), and β-sitosterol (43.4%). The powder was dispersed in water to a solid content concentration of 1 w/v % and then the resulting state was observed. The results are shown in Table 1.

In Examples 1 to 4, the ratio of the plant sterol to phosphatidylcholine was varied from 99:1 to 95:5. It was understood that the state of dispersion in water was good in all of these cases.

### EXAMPLE 5

In accordance with the compositions listed in Table 1, 9.6 g of the plant sterol was heated and melted and then 0.4 g of fractioned and purified lecithin (Trade name: SLP-PC70, Tsuji Oil Mill Co., Ltd., phosphatidylcholine content: 76.2%) was added and dissolved. The resultant was cooled and solidified and then milled with a small mill (HSIANG TAI MACHINERY INDUSTRY, Sample Mill SM-1), so that 10 g of yellow powder was obtained.

The thus obtained powder comprised brassicasterol (5.5%), campesterol (23.7%), stigmasterol (19.4%), and β-sitosterol (43.9%). The powder was dispersed in water to a solid content concentration of 1 w/v % and then the resulting state was observed. The results are shown in Table 1.

In Example 5, 0.4 g of fractioned and purified lecithin (phosphatidylcholine content: 76.2%) was added instead of phosphatidylcholine; that is, 0.3048 g of phosphatidylcholine was added. The ratio of the plant sterol to phosphatidylcholine was almost 97:3, suggesting a good state of dispersion in water.

### EXAMPLE 6

In accordance with the compositions listed in Table 1, procedures were carried out in a manner similar to that in Example 5 except, that the amount of the plant sterol added was 9.5 g and the amount of fractioned and purified lecithin added was 0.5 g. Thus, 10 g of yellow powder was obtained.

The thus obtained powder comprised brassicasterol (5.4%), campesterol (23.5%), stigmasterol (19.2%), and β-sitosterol (43.4%). The powder was dispersed in water to a solid content concentration of 1 w/v % and then the resulting state was observed. The results are shown in Table 1.

In Example 6, 0.381 g of phosphatidylcholine was added. The ratio of the plant sterol to phosphatidylcholine was almost 96:4, suggesting a good state of dispersion in water.

### EXAMPLE 7

In accordance with the compositions listed in Table 1, 9.5 g of the plant sterol was heated and melted, and then 0.5 g of fractioned and purified lecithin (Trade name: Basis LS-60, The Nisshin OilliO Group, Ltd., phosphatidylcholine content: 72%) was added and dissolved. The resultant was cooled and solidified and then milled with a small mill (HSIANG TAI MACHINERY INDUSTRY, Sample Mill SM-1), so that 10 g of yellow powder was obtained.

The thus obtained powder comprised brassicasterol (5.4%), campesterol (23.5%), stigmasterol (19.2%), and β-sitosterol (43.4%). The powder was dispersed in water to a solid content concentration of 1 w/v % and then the resulting state was observed. The results are shown in Table 1.

In Example 7, 0.36 g of phosphatidylcholine was added. The ratio of the plant sterol to phosphatidylcholine was almost 96.35:3.65, suggesting a good state of dispersion in water.

### EXAMPLE 8

In accordance with the compositions listed in Table 1, 9.65 g of the plant sterol was heated and melted and then 0.05 g of phosphatidylcholine and 0.3 g of a sucrose fatty acid ester (Trade name: sugar ester S-1670, Mitsubishi-Kagaku Foods Corporation) were added and dissolved. The resultant was cooled and solidified and then milled with a small mill (HSIANG TAI MACHINERY INDUSTRY, Sample Mill SM-1). Thus, 10 g of white powder was obtained.

The thus obtained powder comprised brassicasterol (5.5%), campesterol (23.8%), stigmasterol (19.5%), and (β-sitosterol (44.1%). The powder was dispersed in water to a solid content concentration of 1 w/v % and then the resulting state was observed. The results are shown in Table 1.

In Example 8, the amount of phosphatidylcholine was lowered and a sucrose fatty acid ester was added instead. The ratio of the plant sterol to phosphatidylcholine and the sucrose fatty acid ester was 96.5:3.5, suggesting a good state of dispersion in water.

### COMPARATIVE EXAMPLE 1

In accordance with the compositions listed in Table 1, procedures were carried out in a manner similar to that in Example 1 except that the amount of the plant sterol added was 8.0 g and the amount, of phosphatidylcholine added was 2.0 g. Thus, 10 g of pale yellow tenacious powder was obtained.

The thus obtained powder comprised brassicasterol (4.6%), campesterol (19.8%), stigmasterol (16.2%), and β-sitosterol (36.6%). The powder was dispersed in water to a solid content concentration of 1 w/v % and then the resulting state was observed. A good state of dispersion in water was observed, but the powder has tenacious property and poor usability.

### COMPARATIVE EXAMPLE 2

In accordance with the compositions listed in Table 1, 9.5 g of the plant sterol was heated and melted and then 0.5 g of phosphatidylserine (phosphatidylserine content: 76.8%) was added and dissolved. The resultant was cooled and solidified and then milled with a small mill (HSIANG TAI MACHINERY INDUSTRY, Sample Mill SM-1), so that 10 g of pale yellow powder was obtained.

The thus obtained powder comprised brassicasterol (5.4%), campesterol (23.5%), stigmasterol (19.2%), and β-sitosterol (43.4%). The powder was dispersed in water to a solid content concentration of 1 w/v % and then the resulting state was observed. The results are shown in Table 1.

In Comparative example 2, phosphatidyl serine was used instead of phosphatidylcholine. The result suggesting poor dispersion in water was obtained.

### COMPARATIVE EXAMPLE 3

In accordance with the compositions listed in Table 1, 9.5 g of the plant sterol was heated and melted and then 0.5 g of phosphatidylglycerol (phosphatidylglycerol content: 71.2%) was added and dissolved. The resultant was cooled and solidified and then milled with a small mill (HSIANG TAI MACHINERY INDUSTRY, Sample Mill SM-1), so that 10 g of pale yellow powder was obtained.

The thus obtained powder comprised brassicasterol (5.4%), campesterol (23.5%), stigmasterol (19.2%), and β-sitosterol (43.4%). The powder was dispersed in water to a solid content concentration of 1 w/v % and then the resulting state was observed. The results are shown in Table 1.

In Comparative example 3, phosphatidylglycerol was used instead of phosphatidylcholine. The result suggesting poor dispersion in water was obtained.

### COMPARATIVE EXAMPLE 4

In accordance with the compositions listed in Table 1, 9.5 g of the plant sterol was heated and melted and then 0.5 g of phosphatidyl ethanolamine (phosphatidyl ethanolamine content: 70.5%) was added and dissolved. The resultant was cooled and solidified and then milled with a small mill (HSIANG TAI MACHINERY INDUSTRY, Sample Mill SM-1), so that 10 g of pale yellow powder was obtained.

The thus obtained powder comprised brassicasterol (5.4%), campesterol (23.5%), stigmasterol (19.2%), and β-sitosterol (43.4%). The powder was dispersed in water to a solid content concentration of 1 w/v % and then the resulting state was observed. The results are shown in Table 1.

In Comparative example 4, phosphatidyl ethanolamine was used instead of phosphatidylcholine. The result suggesting poor dispersion in water was obtained.

### COMPARATIVE EXAMPLE 5

In accordance with the compositions listed in Table 1, 9.5 g of the plant sterol was heated and melted and then 0.5 g of phosphatidic acid (phosphatide acid content: 67.6%) was added and dissolved. The resultant was cooled and solidified and then milled with a small mill (HSIANG TAI MACHINERY INDUSTRY, Sample Mill SM-1), so that 10 g of white powder was obtained.

The thus obtained powder comprised brassicasterol (5.4%), campesterol (23.5%), stigmasterol (19.2%), and β-sitosterol (43.4%). The powder was dispersed in water to a solid content concentration of 1 w/v % and then the resulting state was observed. The results are shown in Table 1.

In Comparative example 5, phosphatidic acid was used instead of phosphatidylcholine. The result suggesting poor dispersion in water was obtained.

### COMPARATIVE EXAMPLE 6

In accordance with the compositions listed in Table 1, 9.5 g of the plant sterol was heated and melted and then 0.5 g of lysophosphatidylcholine (Trade name: SLP-LPC70 Tsuji Oil Mill Co., Ltd., lysophosphatidylcholine content: 68.0%) was added and dissolved. The resultant was cooled and solidified and then milled with a small mill (HSIANG TAI MACHINERY INDUSTRY, Sample Mill SM-1), so that 10 g of yellow powder was obtained.

The thus obtained powder comprised brassicasterol (5.4%), campesterol (23.5%), stigmasterol (19.2%), and β-sitosterol (43.4%). The powder was dispersed in water to a solid content concentration of 1 w/v % and then the resulting state was observed. The results are shown in Table 1.

In Comparative example 6, lysophosphatidylcholine was used instead of phosphatidylcholine. The result suggesting poor dispersion in water was obtained.

### COMPARATIVE EXAMPLE 7

In accordance with the compositions listed in Table 1, 9.5 g of the plant sterol was heated and melted and then 0.5 g of lysophosphatidylglycerol (lysophosphatidylglycerol content: 81.4%) was added and dissolved. The resultant was cooled and solidified and then milled with a small mill (HSIANG TAI MACHINERY INDUSTRY Sample Mill SM-1), so that 10 g of white powder was obtained.

The thus obtained powder comprised brassicasterol (5.4%), campesterol (23.5%), stigmasterol (19.2%), and β-sitosterol (43.4%). The powder was dispersed in water to a solid content concentration of 1 w/v % and then the resulting state was observed. The results are shown in Table 1.

In Comparative example 7, lysophosphatidylglycerol was used instead of phosphatidylcholine. The result suggesting poor dispersion in water was obtained.

TABLE 1

| | Example 1 | Example 2 | Example 3 | Example 4 | Example 5 |
|---|---|---|---|---|---|
| Ingredient name (% by weight) | | | | | |
| Plant sterol (Trade name: PHS-FK) | 99 | 98.5 | 98 | 95 | 96 |
| Phosphatidylcholine | 1 | 1.5 | 2 | 5 | — |
| Fractioned and purified lecithin (Trade name: SLP-PC70) | — | — | — | — | 4 |
| Result | | | | | |
| Brassicasterol | 5.6 | 5.6 | 5.6 | 5.4 | 5.5 |
| Campesterol | 24.5 | 24.3 | 24.2 | 23.5 | 23.7 |
| Stigmasterol | 20.0 | 19.9 | 19.8 | 19.2 | 19.4 |
| β-sitosterol | 45.2 | 45.0 | 44.8 | 43.4 | 43.9 |
| Total | 95.3 | 94.8 | 94.4 | 91.5 | 92.5 |
| State of dispersion in water at | ◯ | ◯ | ◯ | ◯ | ◯ |
| a solid content concentration of 1 w/v % | | | | | |
| | | | | Comparative | Comparative |
| | Example 6 | Example 7 | Example 8 | example 1 | example 2 |
| Ingredient name (% by weight) | | | | | |
| Plant sterol (Trade name: PHS-FK) | 95 | 95 | 96.5 | 80 | 95 |
| Phosphatidylcholine | — | — | 0.5 | 20 | — |
| Fractioned and purified lecithin (Trade name: SLP-PC70) | 5 | — | — | — | — |
| Fractioned and purified lecithin (Trade name: Basis LS-60) | — | 5 | — | — | — |
| Sucrose fatty acid ester (Trade name: sugar ester S-1670) | — | — | 3 | — | — |
| Phosphatidyl serine | — | — | — | — | 5 |
| Result | | | | | |
| Brassicasterol | 5.4 | 5.4 | 5.5 | 4.6 | 5.4 |
| Campesterol | 23.5 | 23.5 | 23.8 | 19.8 | 23.5 |
| Stigmasterol | 19.2 | 19.2 | 19.5 | 16.2 | 19.2 |
| β-sitosterol | 43.4 | 43.4 | 44.1 | 36.6 | 43.4 |
| Total | 91.5 | 91.5 | 92.9 | 77.2 | 91.5 |
| State of dispersion in water at | ◯ | ◯ | ◯ | ◯ | |
| a solid content concentration of 1 w/v % | | | | | |
| | Comparative | Comparative | Comparative | Comparative | Comparative |
| | example 3 | example 4 | example 5 | example 6 | example 7 |
| Ingredient name (% by weight) | | | | | |
| Plant sterol (Trade name: PHS-FK) | 95 | 95 | 95 | 95 | 95 |
| Phosphatidylglycerol | 5 | — | — | — | — |
| Phosphatidyl ethanolamine | — | 5 | — | — | — |
| Phosphatidic acid | — | — | 5 | — | — |
| Lysophosphatidylcholine | — | — | — | 5 | — |
| Lysophosphatidylglycerol | — | — | — | — | 5 |
| Result | | | | | |
| Brassicasterol | 5.4 | 5.4 | 5.4 | 5.4 | 5.4 |
| Campesterol | 23.5 | 23.5 | 23.5 | 23.5 | 23.5 |
| Stigmasterol | 19.2 | 19.2 | 19.2 | 19.2 | 19.2 |
| β-sitosterol | 43.4 | 43.4 | 43.4 | 43.4 | 43.4 |
| Total | 91.5 | 91.5 | 91.5 | 91.5 | 91.5 |
| State of dispersion in water at | | | | | |
| a solid content concentration of 1 w/v % | | | | | |

State of dispersion in water: ∘; dispersed well; and ×: dispersed with difficulty, leading to generation of insoluble matter

### INDUSTRIAL APPLICABILITY

According to the present invention, a plant sterol having a cholesterol-lowering effect can be contained at a high content and dispersed in water, making it possible to use it in wide-ranging fields of food.

All publications, patents, and patent applications cited herein are incorporated herein by reference in their entirety.

1. A composition easily dispersible in water, which is obtained by dissolving phosphatidylcholine in a plant sterol, wherein the content of the plant sterol is 80% by weight or more.

2. The composition according to claim 1, wherein a sucrose fatty acid ester is added.

3. (canceled)

4. The composition according to claim 1, wherein the content of the plant sterol is 95% by weight or more.

5. A method for producing the composition easily dispersed in water according to claim 1, which comprises the following (1) or (2) of:
(1) heating and melting a plant sterol in advance and subsequently adding and dissolving phosphatidylcholine; or
(2) dissolving a plant sterol and phosphatidylcholine in an organic solvent in which both plant sterol and phosphatidylcholine can be dissolved, subsequently distilling off the organic solvent, and then conducting drying.

6. A method for producing the composition easily dispersed in water according to claim 2, which comprises the following (1) or (2) of:
(1) heating and melting a plant sterol in advance and subsequently adding and dissolving phosphatidylcholine and a sucrose fatty acid ester; or
(2) dissolving a plant sterol, phosphatidylcholine, and a sucrose fatty acid ester in an organic solvent in which they can be dissolved together, subsequently distilling off the organic solvent, and then conducting drying.
